(19) United States
(12) Patent Application Publication
(10) Pub. No.: US 2007/0185466 A1
(43) Pub. Date: Aug. 9, 2007

(54) Urine specimen collector assembly

(76) Inventor: Maja Co, Corona, CA (US)

Correspondence Address: MAJA CO, 4156 WINDSPRING STREET, CORONA, CA, 92883, US

(21) Appl. No.: 11/349,771

(22) Filed: Feb. 8, 2006

(51) Int. Cl. A61F 13/15; A61F 5/44

(52) U.S. Cl. 604/349; 604/385.09; 604/385.19; 604/354

(57) ABSTRACT

A disposable urine collector assembly includes a diaper (10) having at the middle an adhesive patch 32 with opening placed over the urinary organ to catch urine and send it to the plastic collection bag (20) situated on the other side of the diaper. The collection bag is made of a small inner bag (24) enclosed in a bigger outer bag (26) with provision for transfer of urine from the inner to the outer bag, however, minimizing reverse or back flow. At the bottom of the outer collection bag is an elongated end with tear-apart section (30) where the urine is withdrawn, transferred to a urine specimen container and sent to the laboratory for analysis. Once the urine sample has been obtained, the collector assembly is detached and properly disposed.

### CROSS-REFERENCE TO RELATED APPLICATIONS

This is a new patent application filed by the present inventor.

### FEDERALLY SPONSORED RESEARCH

Not Applicable

### SEQUENCE LISTING OF PROGRAM

Not applicable

### BACKGROUND OF THE INVENTION

1. Field of Invention

This invention relates to urine collector assembly for all ages of people.

2. Prior Art

Urine collection devices come in different forms and sizes depending on specific purposes and objectives. Disposable urinary catheter systems are available to collect urine by inserting a flexible tube into the urethra in order to withdraw urine from the bladder. The urine flows through the catheter and then directed into a specimen collection bag provided for this purpose where a required amount of urine is separated and collected for laboratory analysis. The excess urine together with the collection bag is disposed accordingly. Considering that the flexible tube is inserted into the urethra, this method of collection especially when the patients are children is painful and may leave traumatic experience to the young ones.

Pediatric urine specimen collectors employs a urine collection bag provided with an adhesive patch at one end having an opening which can surround the male or female organ of children. In this manner, the urine produced is directed to the plastic urine collection bag connected to the adhesive patch. The drawback of the present pediatric urine collector is that during actual use a diaper is placed over the urine collector bag covering the whole part of the urine collector device.

Inasmuch as the urine collector bag is hidden from sight by the diaper, it can not be seen whether the child has already urinated or that a satisfactory amount of urine has been collected. Moreover, the urine in the collection bag is easily contaminated by the stool of the child in case he or she has defecated. In addition, if the urine overflows from the urine collection bag the adhesive patch may be detached with the possibility of urine being contaminated or else insufficient urine is collected. In case the urine sample collected is insufficient or contaminated, it is necessary to repeat the process. Repeating the process will require a new urine collector bag and a diaper and consequently loss of money, time and effort for those concerned.

3. Objects and Advantages

The present invention which is a urine collector assembly wherein the collection bag is over the diaper during use is intended to overcome some problems encountered by the existing system of urine collection. The objects and advantages of the present invention are as follows:
(a) To provide a urine specimen collection device wherein the adhesive patch is fixed to the diaper thus it is less likely that the adhesive patch will be detached ensuring proper urine collection.
(b) To provide a urine specimen collection device comprising of a urine collection bag attached to a diaper in which during actual use the collection bag is over the diaper and away from the body thus it can easily be seen and checked whether sufficient urine has already been collected for the required laboratory sample.
(d) To provide a urine specimen collection device which does not require urethral insertion thus effecting a minimum of discomfort to the patient.
(e) To provide a urine collection bag for all ages of people male and female who are incontinent or non-ambulatory due to accident or other causes.
(f) To provide a urine collection bag with a tear-apart elongated end for ease of transfer of the urine specimen to the laboratory container.

Further objects and advantages are to provide a urine collector assembly which is of unisex design for use by children as well as persons of all ages, which is easy to manipulate and noiseless during use. Still further objects and advantages of my invention will become apparent from a consideration of the ensuing description and drawings.

### SUMMARY

In accordance with the present invention, a disposable urine collector assembly includes a diaper, an adhesive patch with opening at the middle, and plastic collection bag whose elongated end is attached to the adhesive patch. The diaper is between the adhesive patch and the collection bag. The opening of the adhesive patch receives the urine from the human urinary organ then flows through the elongated neck towards the receiving collection bag which is on top of the diaper and can readily be checked of the collected urine. The urine specimen is withdrawn at the bottom of the collection bag, transferred to the urine specimen container for laboratory analysis.

### DRAWINGS

### Figures

In the drawings, closely related figures have the same number but different alphabetic suffixes.

FIG. 1 is a perspective view of the urine collector assembly showing the adhesive patch over the diaper and the collecting bag below.

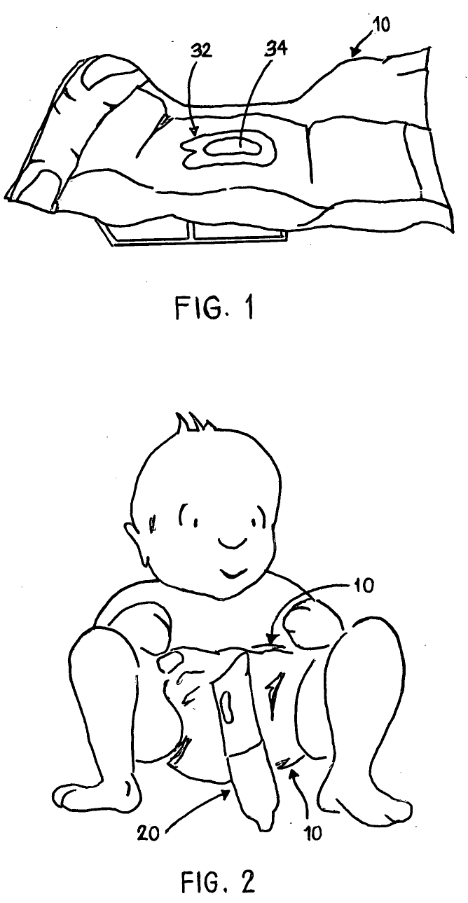

FIG. 2 is a front view of the collector assembly as it appears during actual use showing that the collector bag is atop of the diaper.

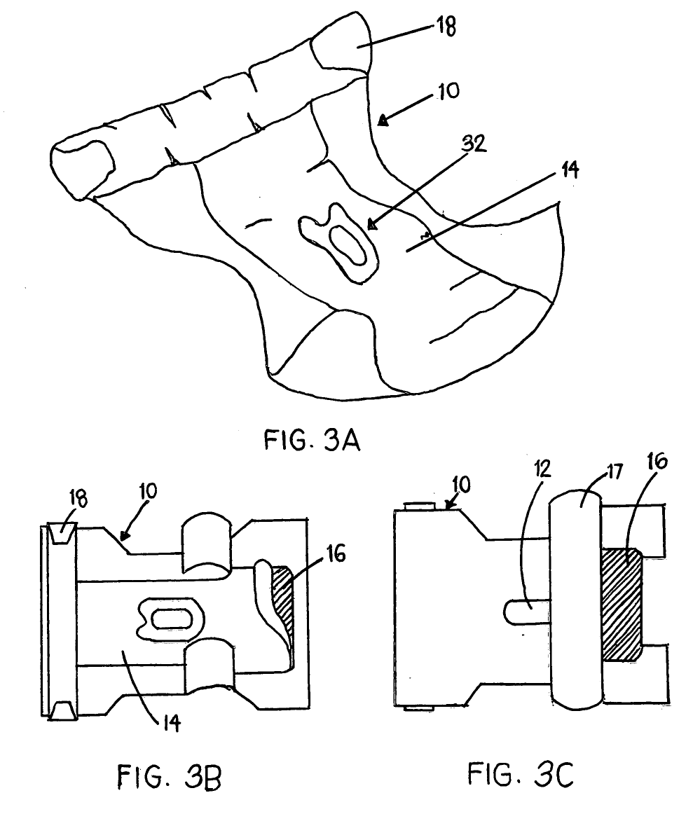

FIG. 3A is the perspective view of the diaper with the adhesive patch. FIG. 3B and FIG. 3C are top side and bottom or opposite side of the diaper, respectively.

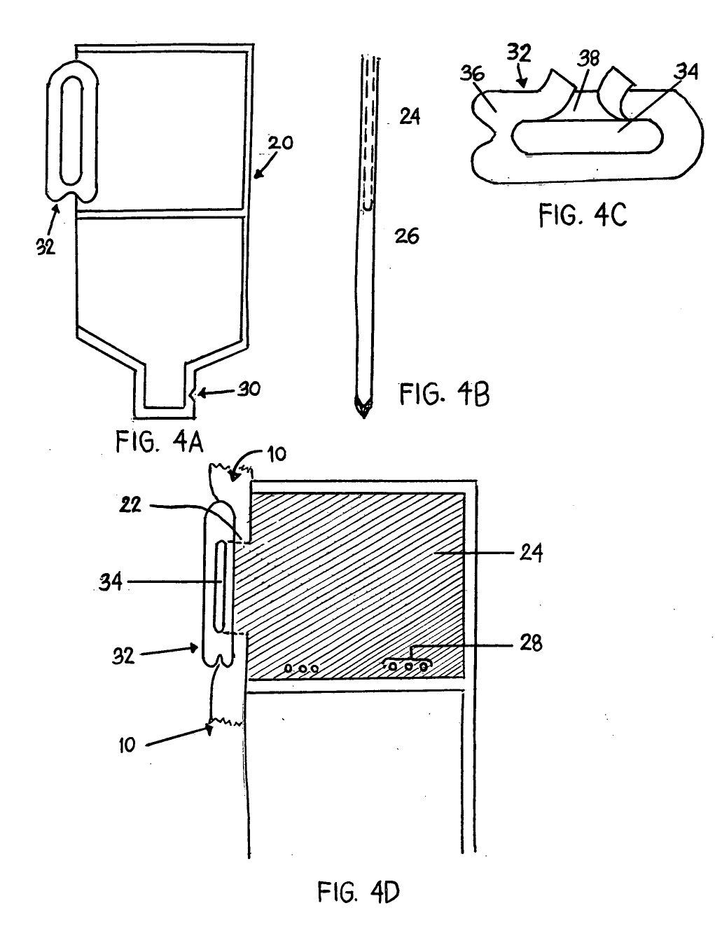

FIG. 4A is an elevational view of the collector bag with the adhesive patch at the top side while the elongated end at the bottom has a tear-apart section where the urine can be withdrawn for transfer to the urine-specimen container. FIG. 4B is the side view of the collection bag showing the inner bag in dotted lines inside the bigger outer bag. FIG. 4C shows top view of the the adhesive patch with a small portion opened to expose the adhesive layer. FIG. 4D shows the relative location of the adhesive patch, diaper and collection bag and the position of the transfer holes in the inner bag.

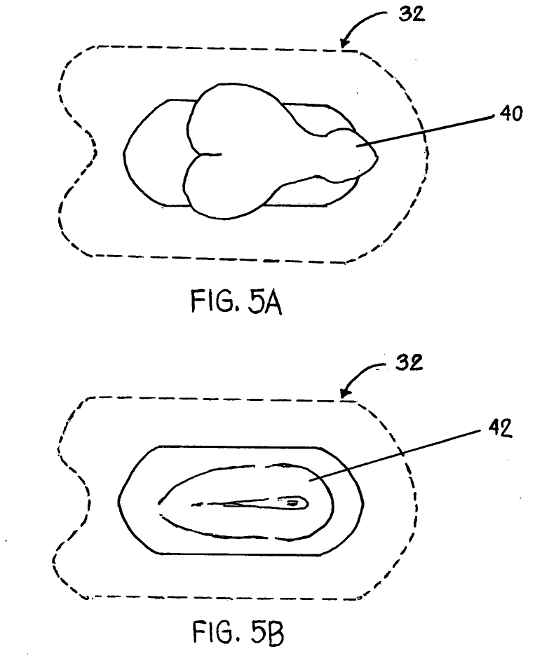

FIGS. 5A and B show the location of the male and female organs relative to the opening of the adhesive patch, respectively.

### REFERENCE NUMERALS

10 diaper
12 diaper opening
14 diaper outer layer
16 diaper inner layer
17 diaper outer layer
18 fastener
20 urine collection bag
22 elongated end
24 inner bag
26 outer bag
28 transfer holes
30 tear-apart section
32 adhesive patch
34 adhesive patch opening
36 adhesive patch outer layer
38 patch inner lining
40 male urinary organ
42 female urinary organ

### DETAILED DESCRIPTION

As shown in FIG. 1, the urine collector assembly comprises a diaper 10, a collector bag 20 partly hidden below the diaper, and an adhesive patch 30 over the diaper. The elongated end 22 of the collector bag passes through a hole 12 in the diaper where at the other side it is attached to one side of the opening 32 of the adhesive patch. The size of the opening of the elongated end is the same as that of the opening of the adhesive patch. The location of the adhesive patch makes it possible for the collection bag to be situated over the diaper and away from the human body during actual use as indicated by FIG. 2.

The diaper 10 (FIG. 3A-C) is rectangular in shape and of flexible material. Sandwiched between its outer layers is an inner layer 16 made of absorbent material preferably cotton. The outer layer 14 where the adhesive patch is attached is of absorbent material while the other layer 17 at the opposite side is waterproof. At one end of the diaper are found fasteners or snap 18 as means for fastening around the waist. The dimension of the diaper with respect to length and width will vary depending on the size of the person using it. At the middle side of the longitudinal section of the diaper where it will be in contact with the human body during use is an adhesive patch 30 which can be pasted around the urinary organ of the user. The opening of the adhesive patch 34 surrounds the urinary organ in such a way that the urine coming out is directed through the elongated end 22 to the inner bag 24 where the elongated end originates.

The collector bag 20 (FIG. 4-A & B) is made of an inner bag 24 completely enclosed by a bigger outer bag 26. An elongated end 22 connected to the inner bag extends outside the outer bag 26 and then passes through the hole 12 in the middle section of the diaper where at the other side of the diaper the adhesive patch is attached to the elongated end. The outer lining of the adhesive patch 36 (FIG. 4-C) is made of pulpy material treated so that it will not stick to the inner layer 38 containing the adhesive layer.

The inner collection bag 24 (FIG. 4D) is made of opposed portions of plastic sheet material that are sealed together around the peripheral edge to define a substantially closed receptacle. The volume that the inner collection bag can contain is nearly half that of the volume of the outer collection bag. At just the bottom section of the inner collection bag and on opposite sides are a number of holes 28 preferably three on each side with diameters big enough to allow passage of urine from the inner collection bag into the outer collection bag but small enough to minimize reverse or back flow of urine from the outer bag to the inner bag.

Enclosing the inner collection bag as well as a small length of the elongated end attached to the inner bag is the outer collection bag 26 made of opposed portions of plastic sheet material that are sealed together around a peripheral edge to define a substantially closed bag. At the lower part of the outer collection bag 26 is an elongated end 30 with a tear-apart section. Once opened it will allow urine specimen to be transferred to the laboratory container for analysis.

The positioning of the adhesive patch during actual use for males 40 is in FIG. 5A while that for females 42 is in FIG. 5B. The opening of the adhesive patch for males is sufficient to contain the whole penis including the testicles while that of the female is restricted to a part of the skin just outside the vulva. The area that the adhesive patch will occupy is sufficient to let it stick firmly during use.

I claim:

1. A disposable urine collector assembly comprising:
(a) a urine collection bag having sufficient size to contain urine that will be withdrawn for laboratory testing,
(b) an adhesive patch having an opening at the middle connected to the elongated end of the collection bag to catch urine from the human urinary organ,
(c) a diaper to hold firmly the adhesive patch so that it will not be detached during use.

2. The collector assembly of claim 1 wherein the body of said urine collection bag is made of opposed portions of plastic sheet material that are sealed together around the peripheral edge to define a substantially closed receptacle and rendered aseptic during manufacture.

3. The collector assembly of claim 1 wherein said collection bag comprises an inner bag enclosed completely by a bigger outer bag and provided with holes for transfer of urine from the said inner bag to the said outer bag.

4. The collector assembly of claim 3 wherein the said inner bag has an elongated end extending outside the said collection bag and connected to the opening of the said adhesive patch for urine to pass through.

5. The collector assembly of claim 3 wherein the said outer bag has an elongated end at the bottom with a tear-away section for easy transfer of urine to the specimen container to be sent to the laboratory for analysis.

6. The collector assembly of claim 1 wherein said adhesive patch has opening to surround the human urinary organ and that said opening is nearly of the same size as that of the said elongated end of the said bigger bag.

7. The collector assembly of claim 1 wherein said diaper is rectangular in shape made of flexible material and its inner layer made of absorbent material.

8. The collector assembly of claim 1 wherein said diaper has an opening at the middle where the said elongated end of the said collection bag passes though such that the said diaper is positioned between the adhesive patch and the said collection bag.

9. The collector assembly of claim 1 wherein during actual use the said collection bag is outside the said diaper thus it will be easy to check whether sufficient urine has been collected for transfer to the specimen container.
